(19) United States
(12) Patent Application Publication
(10) Pub. No.: US 2019/0170884 A1
(43) Pub. Date: Jun. 6, 2019

(54) IMAGING PANEL AND METHOD FOR PRODUCING SAME

(71) Applicant: Sharp Kabushiki Kaisha, Sakai City, Osaka (JP)

(72) Inventor: KATSUNORI MISAKI, Yonago-shi, Tottori (JP)

(21) Appl. No.: 16/322,899

(22) PCT Filed: Jul. 31, 2017
(86) PCT No.: PCT/JP2017/027762

§ 371 Date: Feb. 1, 2019

(30) Foreign Application Priority Data
Aug. 3, 2016 (JP) 2016-153025

(51) Int. Cl. G01T 1/20; A61B 6/00; H04N 5/32; H01L 31/08; H01L 27/146; G01N 23/04

(52) U.S. Cl. CPC G01T 1/2018; A61B 6/00; G01N 23/04; H01L 31/085; H01L 27/14643; H04N 5/32

(57) ABSTRACT

Provided is an X-ray imaging panel in which leakage current in a photoelectric conversion layer can be suppressed, and a method for producing the same. An imaging panel 1 generates an image based on scintillation light obtained from X-rays transmitted through an object. The imaging panel 1 includes, on a substrate 101, a thin film transistor 13, an insulating film 103 covering the thin film transistor 13, a photoelectric conversion layer 15 that converts the scintillation light into charges, an upper electrode 14b, a lower electrode 14a connected with the thin film transistor 13, and an upper electrode protection film 18 covering the upper electrode 14b. Ends of the upper electrode 14b are arranged in such a manner that each end thereof is arranged on an inner side of the photoelectric conversion layer 15 with respect to a corresponding end of the photoelectric conversion layer 15. Ends of the upper electrode protection film 18 are arranged in such a manner that each end thereof is arranged between a corresponding end of the upper electrode 14b and a corresponding end of the photoelectric conversion layer 15.

### TECHNICAL FIELD

The present invention relates to an imaging panel and a method for producing the same.

### BACKGROUND ART

An X-ray imaging device that picks up an X-ray image with an imaging panel that includes a plurality of pixel portions is known. In such an X-ray imaging device, irradiated X-rays are converted into charges by, for example, p-intrinsic-n (PIN) photodiodes. Converted charges are read out by thin film transistors (hereinafter also referred to as TFTs) that are caused to operate, the TFTs being provided in the pixel portions. With the charges being read out in this way, an X-ray image is obtained.

JP-A-2014-78651 discloses a photoelectric conversion device that is such an X-ray imaging device. In this photoelectric conversion device, a photoelectric conversion layer is provided on the lower electrodes, upper electrodes are provided on the photoelectric conversion layer, and a protection film covering side surfaces of the photoelectric conversion layer is provided on the upper electrodes.

### SUMMARY OF THE INVENTION

### Problem to be Solved by the Invention

The photodiode of the X-ray imaging device as described above can be formed by forming semiconductor films of an n-layer, an i-layer, and a p-layer that compose the photoelectric conversion layer, sequentially on the lower electrodes, forming the upper electrodes on the p-layer, applying a resist so that the resist covers the upper electrodes, and etching the semiconductor films. After etching, in order to suppress leakage current in the photoelectric conversion layer, the side surfaces of the photoelectric conversion layer are subjected to a reduction treatment with hydrogen fluoride in some cases, in a case where this reduction treatment is carried out after the resist is removed, the upper electrodes are dissolved by the reduction treatment, and metal ions adhere to the side surfaces of the photoelectric conversion layer. In a case where the reduction treatment is carried out before the resist is removed, organic substances adhere to the side surfaces of the photoelectric conversion layer due to a removing liquid that is used when the resist is removed. If metal ions or organic substances adhere to the side surfaces of the photoelectric conversion layer in this way, it is impossible to achieve an effect of suppressing leakage current even if the reduction treatment using hydrogen fluoride is carried out with respect to the side surfaces of the photoelectric conversion layer.

It is an object of the present invention to provide an X-ray imaging panel in which leakage current in the photoelectric conversion layer can be suppressed, and to provide a method for producing the same.

An imaging panel of the present invention with which the above-described problem is solved is an imaging panel that generates an image based on scintillation light that is obtained from X-rays transmitted through an object, and the imaging panel includes: a substrate; a thin film transistor that is formed on the substrate; an insulating film that covers the thin film transistor; a photoelectric conversion layer that is provided on the insulating film, and converts the scintillation light into charges; an upper electrode that is provided on the photoelectric conversion layer; a lower electrode that is provided under the photoelectric conversion layer, and is connected with the thin film transistor; and an upper electrode protection film that covers the upper electrode, above the photoelectric conversion layer, wherein ends of the upper electrode are arranged in such a manner that each end thereof is arranged on an inner side of the photoelectric conversion layer with respect to a corresponding end of the photoelectric conversion layer, and ends of the upper electrode protection film are arranged in such a manner that each end thereof is arranged between a corresponding end of the upper electrode and a corresponding end of the photoelectric conversion layer.

With the present invention, leakage current in the photoelectric conversion layer can be suppressed.

### BRIEF DESCRIPTION OF DRAWINGS

FIG. 1 schematically illustrates a schematic configuration of an X-ray imaging device in an embodiment.

FIG. 2 schematically illustrates a schematic configuration of an imaging panel illustrated in FIG. 1.

FIG. 3 is an enlarged plan view illustrating one pixel portion of an imaging panel 1 illustrated in FIG. 2.

FIG. 4A is a cross-sectional view of the pixel illustrated in FIG. 3, taken along the line A-A.

FIG. 4B is an enlarged cross-sectional view of a part including an upper electrode protection film illustrated in FIG. 4A.

FIG. 5A is a cross-sectional view illustrating a step of forming a first insulating film on a gate insulating film and a TFT formed on a substrate.

FIG. 5B is a cross-sectional view illustrating a step of forming a contact hole CH1 in the first insulating film illustrated in FIG. 5A.

FIG. 5C is a cross-sectional view illustrating a step of forming a second insulating film on the first insulating film illustrated in FIG. 5B.

FIG. 5D is a cross-sectional view illustrating a step of forming an opening in the second insulating film, on the contact hole CH1 illustrated in FIG. 5C.

FIG. 5E is a cross-sectional view illustrating a step of forming a metal film on the second insulating film illustrated in FIG. 5D.

FIG. 5F is a cross-sectional view illustrating a step of patterning the metal film illustrated in FIG. 5E so as to form a lower electrode connected with a drain electrode via the contact hole CH1.

FIG. 5G is a cross-sectional view illustrating a step of forming n-type amorphous semiconductor layer, an intrinsic amorphous semiconductor layer, and a p-type amorphous semiconductor layer so that these layers cover the lower electrode illustrated in FIG. 5F, and forming a transparent conductive film on the p-type amorphous semiconductor layer.

FIG. 5H is a cross-sectional view illustrating a step of patterning the transparent conductive film illustrated in FIG. 5G so as to form an upper electrode.

FIG. 5I is a cross-sectional view illustrating a step of forming an insulating film so that the insulating film covers the upper electrode illustrated in FIG. 5H.

FIG. 5J is a cross-sectional view illustrating a step of patterning the insulating film, the n-type amorphous semiconductor layer, the intrinsic amorphous semiconductor layer, and the p-type amorphous semiconductor layer illustrated in FIG. 5I so as to form a photoelectric conversion layer and an upper electrode protection film.

FIG. 5K is a cross-sectional view illustrating a state after removing a resist used in the step of FIG. 5J and carrying out a reduction treatment in which hydrogen fluoride is applied to the surface of the photoelectric conversion layer.

FIG. 5L is a cross-sectional view illustrating a step of forming a third insulating film on the upper electrode protection film illustrated in FIG. 5K.

FIG. 5M is a cross-sectional view illustrating a step of forming a contact hole CH2 that passes through the third insulating film and the upper electrode protection film illustrated in FIG. 5L.

FIG. 5N is a cross-sectional view illustrating a step of forming a fourth insulating film on the third insulating film illustrated in FIG. 5M, and forming an opening in the fourth insulating film, on the contact hole CH2.

FIG. 5O is a cross-sectional view illustrating a step of forming a metal film on the fourth insulating film illustrated in FIG. 5N.

FIG. 5P is a cross-sectional view illustrating a step of forming a bias line by patterning the metal film illustrated in FIG. 5O.

FIG. 5O is a cross-sectional view illustrating a step of forming a transparent conductive film so that the transparent conductive film covers the bias line illustrated in FIG. 5P.

FIG. 5R is a cross-sectional view illustrating a step of patterning the transparent conductive film illustrated in FIG. 5Q.

FIG. 5S is a cross-sectional view illustrating a step of forming a fifth insulating film so that the fifth insulating film covers the transparent conductive film illustrated in FIG. 5R.

FIG. 5T is a cross-sectional view illustrating a step of forming a sixth insulating film on the fifth insulating film illustrated in FIG. 5S.

FIG. 6 is a cross-sectional view illustrating an imaging panel after a reduction treatment in Embodiment 3 is carried out.

### MODE FOR CARRYING OUT THE INVENTION

An imaging panel according to one embodiment of the present invention is an imaging panel that generates an image based on scintillation light that is obtained from X-rays transmitted through an object, and the imaging panel includes: a substrate; a thin film transistor that is formed on the substrate; an insulating film that covers the thin film transistor; a photoelectric conversion layer that is provided on the insulating film, and converts the scintillation light into charges; an upper electrode that is provided on the photoelectric conversion layer; a lower electrode that is provided under the photoelectric conversion layer, and is connected with the thin film transistor; and an upper electrode protection film that covers the upper electrode, above the photoelectric conversion layer, wherein ends of the upper electrode are arranged in such a manner that each end thereof is arranged on an inner side of the photoelectric conversion layer with respect to a corresponding end of the photoelectric conversion layer, and ends of the upper electrode protection film are arranged in such a manner that each end thereof is arranged between a corresponding end of the upper electrode and a corresponding end of the photoelectric conversion layer (the first configuration).

According to the first configuration, the upper electrode protection film is formed on the upper electrode. Ends of the upper electrode are arranged in such a manner that each end thereof is arranged on an inner side of the photoelectric conversion layer with respect to a corresponding end of the photoelectric conversion layer, and ends of the upper electrode protection film are arranged in such a manner that each end thereof is arranged between a corresponding end of the upper electrode and a corresponding end of the photoelectric conversion layer. In other words, the upper electrode is covered with the upper electrode protection film, on the photoelectric conversion layer. As compared with a case where the upper electrode protection film is not provided, it is therefore less likely that the photoelectric conversion layer would be affected by a reduction treatment using hydrogen fluoride, which is intended to suppress leakage current in the photoelectric conversion layer, or by a resist removing liquid that is used when the photoelectric conversion layer is formed. It is therefore unlikely that organic substances or metal ions would adhere to the surface of the photoelectric conversion layer, which results in that leakage current in the photoelectric conversion layer can be suppressed.

The first configuration may be such that the upper electrode protection film is made of silicon nitride (the second configuration).

With the second configuration, leakage current in the photoelectric conversion layer can be suppressed, and at the same time, the adhesiveness with the upper electrode can be improved.

The first configuration may be such that the upper electrode protection film is made of silicon oxide (the third configuration).

With the third configuration, leakage current in the photoelectric conversion layer can be suppressed.

The first configuration may be such that the upper electrode protection film is made of silicon oxide nitride (the fourth configuration).

With the fourth configuration, leakage current in the photoelectric conversion layer can be suppressed.

A method for producing an imaging panel according to one embodiment of the present invention is a method for producing an imaging panel that generates an image based on scintillation light that is obtained from X-rays transmitted through an object, and the producing method includes: forming a thin film transistor on a substrate; forming a first insulating film and a second insulating film on the thin film transistor; forming a first contact hole on a drain electrode of the thin film transistor so that the first contact hole passes through the first insulating film and the second insulating film; forming, on the second insulating film, a first transparent electrode film as a lower electrode that is connected with the drain electrode through the first contact hole; forming, on the first transparent electrode film, a first semiconductor layer of a first conductive type as a photoelectric conversion layer, an intrinsic amorphous semiconductor layer, and a second semiconductor layer of a second conductive type that is opposite to the first conductive type of the first semiconductor layer, in the stated order; forming an upper electrode on the second semiconductor layer; forming an insulating film as an upper electrode protection film, on the upper electrode; applying a resist on the insulating film, and etching the insulating film, the first semiconductor layer, the intrinsic amorphous semiconductor layer, and the second semiconductor layer, so as to form the photoelectric conversion layer and the upper electrode protection film; removing the resist, and thereafter, carrying out a reduction treatment with respect to a surface of the photoelectric conversion layer; forming a third insulating film that covers the upper electrode protection film, after the reduction treatment; forming a second contact hole on the upper electrode so that the second contact hole passes through the third insulating film and the upper electrode protection film; forming a fourth insulating film on the third insulating film except for a portion of the second contact hole; forming a signal line for supplying a bias voltage, on the fourth insulating film; forming, on the fourth insulating film, a transparent conductive film that connects the signal line and the upper electrode with each other through the second contact hole; and forming a fifth insulating film that covers the transparent conductive film (the fifth configuration).

According to the fifth configuration, after the photoelectric conversion layer is formed and the resist is removed, the surface of the photoelectric conversion layer is subjected to the reduction treatment. As compared with a case where the reduction treatment is applied before the resist is removed, it is therefore unlikely that the surface of the photoelectric conversion layer would be contaminated with organic substances. Further, since the upper electrode protection film is formed on the upper electrode, even if the reduction treatment is carried out after the resist is removed, such a phenomenon does not occur that metal ions generated as a result of dissolution of the upper electrode would adhere to a surface of the photoelectric conversion layer. This consequently makes it possible to produce an imaging panel in which leakage current in the photoelectric conversion layer is suppressed.

The fifth configuration may be such that, as the reduction treatment, a reduction treatment using hydrogen fluoride is carried out (the sixth configuration).

With the sixth configuration, leakage current in the photoelectric conversion layer can be suppressed.

The sixth configuration may be such that, after the reduction treatment using hydrogen fluoride is carried out, before the third insulating film is formed, a hydrogen-gas-containing plasma treatment is carried out (the seventh configuration).

With the seventh configuration, even if a hydrogen-gas-containing plasma treatment is carried out before the third insulating film is formed, the upper electrode therefore is not affected by the plasma treatment since it is covered with the upper electrode protection film, and the transmittance of the upper electrode therefore does not decrease. As a result, without decreasing the light receiving sensitivity of the photoelectric conversion layer, the effect of suppressing leakage current in the photoelectric conversion layer can be improved.

The fifth configuration may be such that, as the reduction treatment, a reduction treatment using hydrogen gas is carried out (the eighth configuration).

With the eighth configuration, even if a hydrogen-gas-containing plasma treatment is carried out after the resist is removed, the upper electrode therefore is not affected by the plasma treatment since it is covered with the upper electrode protection film, and the transmittance of the upper electrode therefore does not decrease. As a result, without decreasing the light receiving sensitivity of the photoelectric conversion layer, leakage current in the photoelectric conversion layer can be suppressed.

The following description describes embodiments of the present invention in detail while referring to the drawings. Identical or equivalent parts in the drawings are denoted by the same reference numerals and descriptions of the same are not repeated.

### Embodiment 1

### (Configuration)

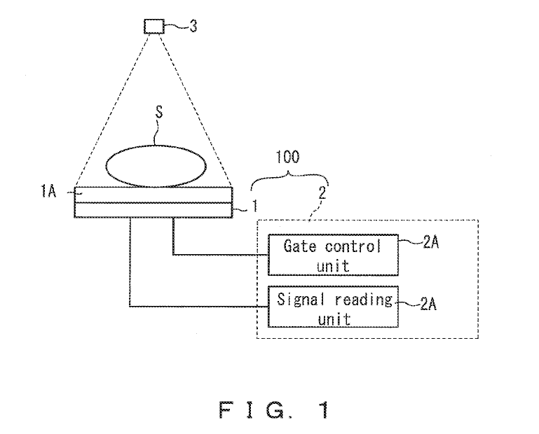

FIG. 1 is a schematic diagram illustrating an X-ray imaging device in the present embodiment. The X-ray imaging device 100 includes an imaging panel 1 and a control unit 2. The control unit 2 includes a gate control unit 2A and a signal reading unit 2B. X-rays are projected from the X-ray source 3 to an object S, and X-rays transmitted through the object S are converted into fluorescence (hereinafter referred to as scintillation light) by a scintillator 1A provided above the imaging panel 1. The X-ray imaging device 100 acquires an X-ray image by picking up the scintillation light with the imaging panel 1 and the control unit 2.

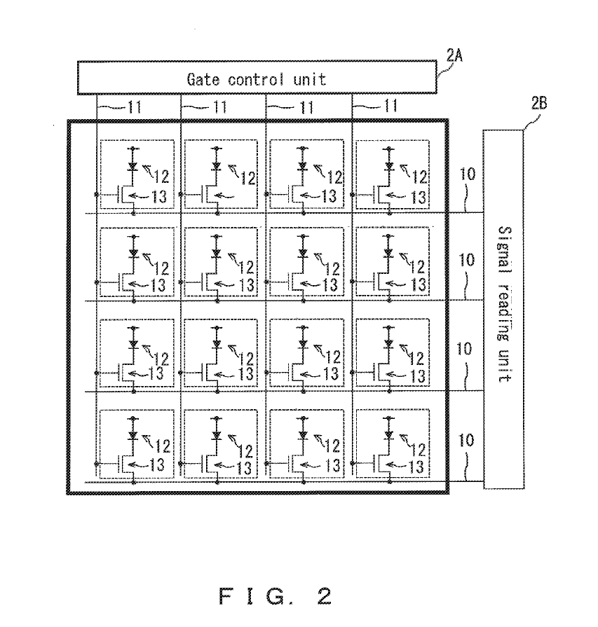

FIG. 2 is a schematic diagram illustrating a schematic configuration of the imaging panel 1. As illustrated in FIG. 2, a plurality of source lines 10, and a plurality of gate lines 11 intersecting with the source lines 10 are formed in the imaging panel 1. The gate lines 11 are connected with the gate control unit 2A, and the source lines 10 are connected with the signal reading unit 2B.

The imaging panel 1 includes TFTs 13 connected to the source lines 10 and the gate lines 11, at positions at which the source lines 10 and the gate lines 11 intersect. Further, photodiodes 12 are provided in areas surrounded by the source lines 10 and the gate lines 11 (hereinafter referred to as pixels). In each pixel, scintillation light obtained by converting X-rays transmitted through the object S is converted by the photodiode 12 into charges according to the amount of the light.

The gate lines 11 in the imaging panel 1 are sequentially switched by the gate control unit 2A into a selected state, and the TFT 13 connected to the gate line 11 in the selected state is turned ON. When the TFT 13 is turned ON, a signal according to the charges obtained by the conversion by the photodiode 12 is output through the source line 10 to the signal reading unit 2B.

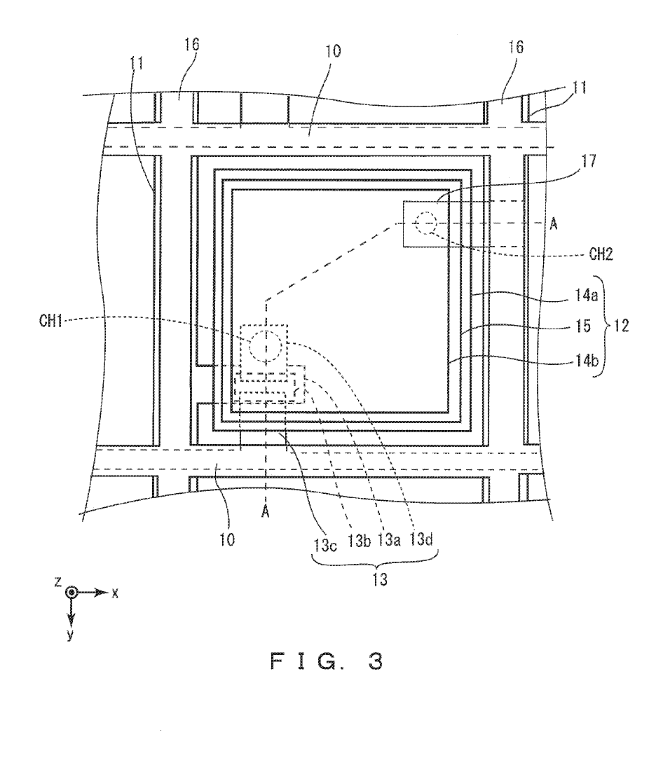

FIG. 3 is an enlarged plan view of one pixel portion of the imaging panel 1 illustrated in FIG. 2. As illustrated in FIG. 3, in the pixel surrounded by the gate line 11 and the source line 10, a lower electrode 14a, a photoelectric conversion layer 15, and an upper electrode 14b that compose the photodiode 12 are arranged so as to overlap with one another. Further, a bias line 16 is arranged so as to overlap with the gate line 11 and the source line 10 when viewed in a plan view. The bias line 16 supplies a bias voltage to the photodiode 12. The TFT 13 includes a gate electrode 13a integrated with the gate line 11 a semiconductor activity layer 13b, a source electrode 13c integrated with the source line 10, and a drain electrode 13d. In the pixel, a contact hole CH1 for connecting the drain electrode 13d and the lower electrode 14a with each other is provided. Further, in the pixel, a transparent conductive film 17 is provided so as to overlap with the bias line 16, and a contact hole CH2 for connecting the transparent conductive film 17 and the upper electrode 14b with each other is provided.

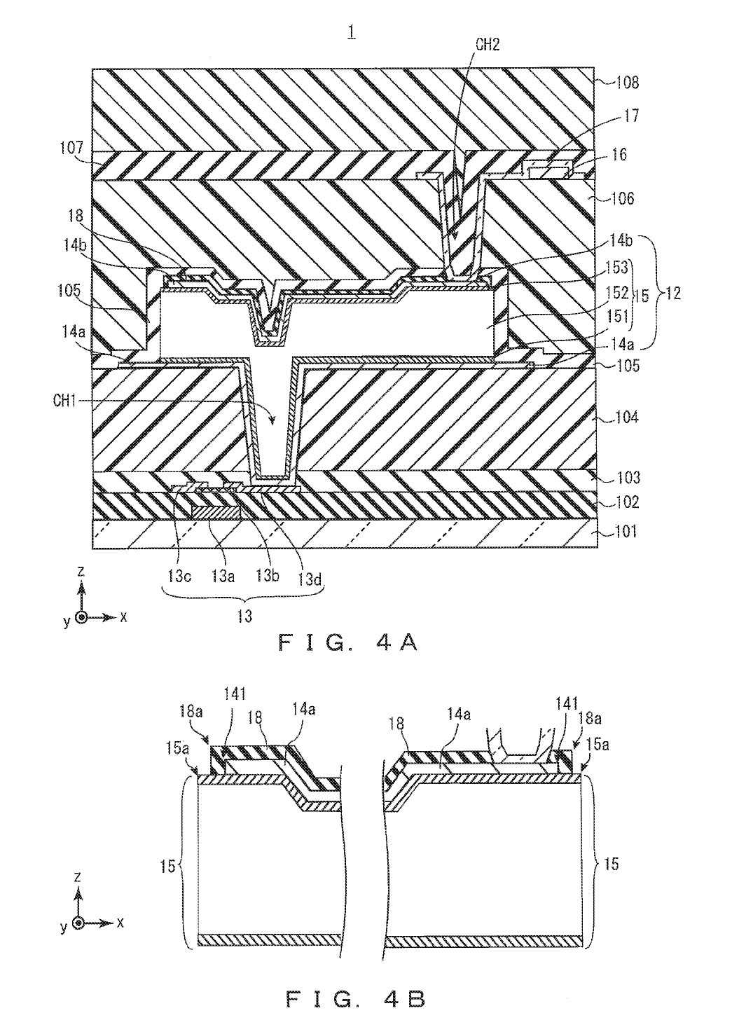

Here, FIG. 4A illustrates a cross-sectional view of the pixel illustrated in FIG. 3 taken along line A-A. As illustrated in FIG. 4A, the TFT 13 is formed on the substrate 101. The substrate 101 is a substrate having insulating properties, such as a glass substrate, a silicon substrate, a plastic substrate having heat-resisting properties, or a resin substrate.

On the substrate 101, the gate electrode 13a integrated with the gate line 11 is formed. The gate electrode 13a and the gate line 11 are made of, for example, a metal such as aluminum (Al), tungsten (W), molybdenum (Mo), molybdenum nitride (MoN), tantalum (Ta), chromium (Cr), titanium (Ti), or copper (Cu), an alloy of any of these metals, or a metal nitride of these metals. In the present embodiment, the gate electrode 13a and the gate line 11 have a laminate structure in which a metal film made of molybdenum nitride and a metal film made of aluminum are laminated in this order. Regarding thicknesses of these metal films, for example, the metal film made of molybdenum nitride has a thickness of 100 nm, and the metal film made of aluminum has a thickness of 300 nm.

The gate insulating film 102 is formed on the substrate 101, and covers the gate electrode 13a. The gate insulating film 102 may be formed with, for example, silicon oxide (SiOₓ), silicon nitride (SiNₓ), silicon oxide nitride (SiOₓN_{y})(x>y), or silicon nitride oxide (SiNₓO_{y})(x>y). In the present embodiment, the gate insulating film 102 is formed with a laminate film obtained by laminating silicon oxide (SiOₓ) and silicon nitride (SiNₓ) in the order, and regarding the thicknesses of these films, the film of silicon oxide (SiOₓ) has a thickness of 50 nm, and the film of silicon nitride (SiNₓ) has a thickness of 400 nm.

The semiconductor activity layer 13b, as well as the source electrode 13c and the drain electrode 13d connected with the semiconductor activity layer 13b are formed on the gate electrode 13a with the gate insulating film 102 being interposed therebetween.

The semiconductor activity layer 13b is formed in contact with the gate insulating film 102. The semiconductor activity layer 13b is made of an oxide semiconductor. For forming the oxide semiconductor, for example, the following material may be used: InGaO₃(ZnO)₅; magnesium zinc oxide (MgₓZn₁₋ₓO); cadmium zinc oxide (CdₓZn₁₋ₓO); cadmium oxide (CdO); or an amorphous oxide semiconductor containing indium (In), gallium (Ga), and zinc (Zn) at a predetermined ratio. In the present embodiment, the semiconductor activity layer 13b is made of an amorphous oxide semiconductor containing indium (In), gallium (Ga), and zinc (Zn) at a predetermined ratio, and has a thickness of, for example, 70 nm.

The source electrode 13c and the drain electrode 13d are formed in contact with the semiconductor activity layer 13b and the gate insulating film 102. The source electrode 13c is integrated with the source line 10. The drain electrode 13d is connected with the lower electrode 14a through the contact hole CH1.

The source electrode 13c and the drain electrode 13d are formed in the same layer, and are made of, for example, a metal such as aluminum (Al), tungsten (W), molybdenum (Mo), tantalum (Ta), chromium (Cr), titanium (Ti), or copper (Cu), or alternatively, an alloy of any of these, or a metal nitride of any of these. Further, as the material for the source electrode 13c and the drain electrode 13d, the following material may be used: a material having translucency such as indium tin oxide (ITO), indium zinc oxide (IZO), indium tin oxide (ITSO) containing silicon oxide, indium oxide (In₂O₃), tin oxide (SnO₂), zinc oxide (ZnO), or titanium nitride; or a material obtained by appropriately combining any of these.

The source electrode 13c and the drain electrode 13d may be, for example, a laminate of a plurality of metal films. More specifically, the source electrode 13c, the source line 10, and the drain electrode 13d have a laminate structure in which a metal film made of molybdenum nitride (MoN), a metal film made of aluminum (Al), and a metal film made of molybdenum nitride (MoN) are laminated in this order. Regarding the thicknesses of the films, the metal film in the lower layer, which is made of molybdenum nitride (MoN), has a thickness of 100 nm, the metal film made of aluminum (Al) has a thickness of 500 nm, and the metal film in the upper layer, which is made of molybdenum nitride (MoN), has a thickness of 50 nm.

A first insulating film 103 is provided so as to cover the source electrode 13c and the drain electrode 13d. The first insulating film 103 may have a single layer structure made of silicon oxide (SiO₂) or silicon nitride (SiN), or a laminate structure obtained by laminating silicon nitride (SiN) and silicon oxide (SiO₂) in this order.

On the first insulating film 103, a second insulating film 104 is formed. The second insulating film 104 is made of an organic transparent resin, for example, acrylic resin or siloxane-based resin, has a thickness of, for example, 2.5 μm.

On the drain electrode 13d, the contact hole CH1 is formed, which passes through the second insulating film 104 and the first insulating film 103.

On the second insulating film 104, the lower electrode 14a, which is connected with the drain electrode 13d through the contact hole CH1, is formed. The lower electrode 14a is formed with, for example, a metal film containing molybdenum nitride (MoN), and has a thickness of, for example, 200 nm.

On the lower electrode 14a, the photoelectric conversion layer 15 is formed. The photoelectric conversion layer 15 is composed of the n-type amorphous semiconductor layer 151, the intrinsic amorphous semiconductor layer 152, and the p-type amorphous semiconductor layer 153, which are laminated in the order.

The n-type amorphous semiconductor layer 151 is made of amorphous silicon doped with an n-type impurity (for example, phosphorus). The n-type amorphous semiconductor layer 151 has a thickness of, for example, 30 nm.

The intrinsic amorphous semiconductor layer 152 is made of intrinsic amorphous silicon. The intrinsic amorphous semiconductor layer 152 is formed in contact with the n-type amorphous semiconductor layer 151. The intrinsic amorphous semiconductor layer has a thickness of, for example, 1000 nm.

The p-type amorphous semiconductor layer 153 is made of amorphous silicon doped with a p-type impurity (for example, boron). The p-type amorphous semiconductor layer 153 is formed in contact with the intrinsic amorphous semiconductor layer 152. The p-type amorphous semiconductor layer 153 has a thickness of, for example, 5 nm.

On the p-type amorphous semiconductor layer 153, the upper electrode 14b is formed. The upper electrode 14b is made of, for example, indium tin oxide (ITO), and has a thickness of, for example, 70 nm.

On the p-type amorphous semiconductor layer 153, an insulating film 18 (hereinafter referred to as an upper electrode protection film) is formed so as to cover the upper electrode 14b. The upper electrode protection film 18 is, for example, an inorganic insulating film made of silicon oxide (SiO₂), and has a thickness of, for example, 100 nm.

FIG. 4B is an enlarged view illustrating a part of the photoelectric conversion layer 15, the upper electrode 14b, and the upper electrode protection film 18 illustrated in FIG. 4A. An X-axis direction end 18a of the upper electrode protection film 18 in the present embodiment is arranged between an X-axis direction end 141 of the upper electrode 14b, and an X-axis direction end 15a of the photoelectric conversion layer 15.

Referring back to FIG. 4A, a third insulating film 105 is formed on the second insulating film 104 so as to cover the photodiode 12 and the upper electrode protection film 18. The third insulating film 105 is, for example, an inorganic insulating film made of silicon nitride (SiN), and has a thickness of, for example, 300 nm.

In the third insulating film 105 and the upper electrode protection film 18, a contact hole CH2 is formed at a position that overlaps with the upper electrode 14b.

On the third insulating film 105, in an area thereof except for the contact hole CH2, a fourth insulating film 106 is formed. The fourth insulating film 106 is formed with an organic transparent resin made of, for example, acrylic resin or siloxane-based resin, and has a thickness of, for example, 2.5 μm.

On the fourth insulating film 106, the bias line 16 is formed. Further, on the fourth insulating film 106, the transparent conductive film 17 is formed so as to overlap with the bias line 16. The transparent conductive film 17 is in contact with the upper electrode 14b at the contact hole CH2. The bias line 16 is connected to the control unit 2 (see FIG. 1). The bias line 16 applies a bias voltage through the contact hole CH2 to the upper electrode 14b, the bias voltage being input from the control unit 2. The bias line 16 has a laminate structure that is obtained by laminating, for example, a metal film made of molybdenum nitride (MoN), a metal film made of aluminum (Al), and a metal film made of titanium (Ti) in this order. The films of molybdenum nitride (MoN), aluminum (Al), and titanium (Ti) have thicknesses of, for example, 100 nm, 300 nm, and 50 nm, respectively.

On the fourth insulating film 106, a fifth insulating film 107 is formed so as to cover the transparent conductive film 17. The fifth insulating film 107 is an inorganic insulating film made of, for example, silicon nitride (SiN), and has a thickness of, for example, 200 nm.

On the fifth insulating film 107, a sixth insulating film 108 is formed. The sixth insulating film 108 is made of, for example, an organic transparent resin such as acrylic resin or siloxane-based resin, and has a thickness of, for example, 2.0 μm.

### (Method for Producing Imaging Panel 1)

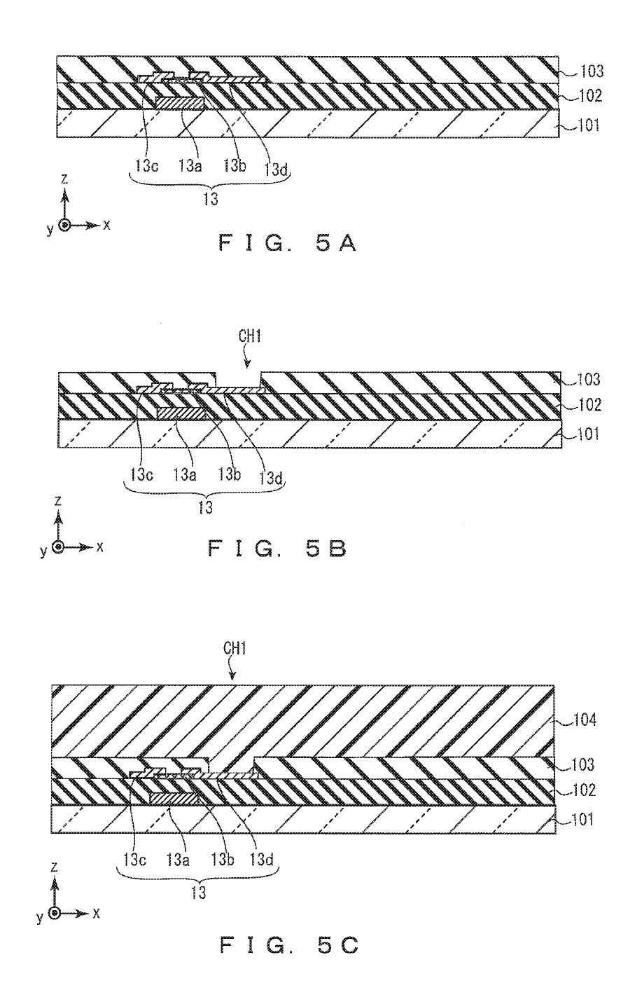

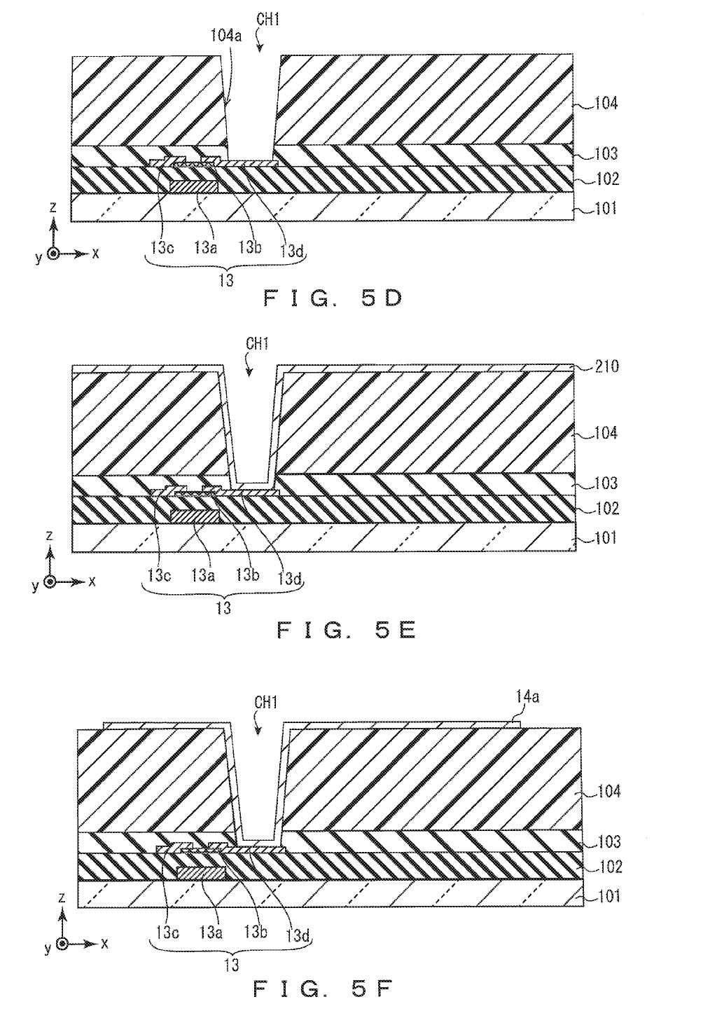

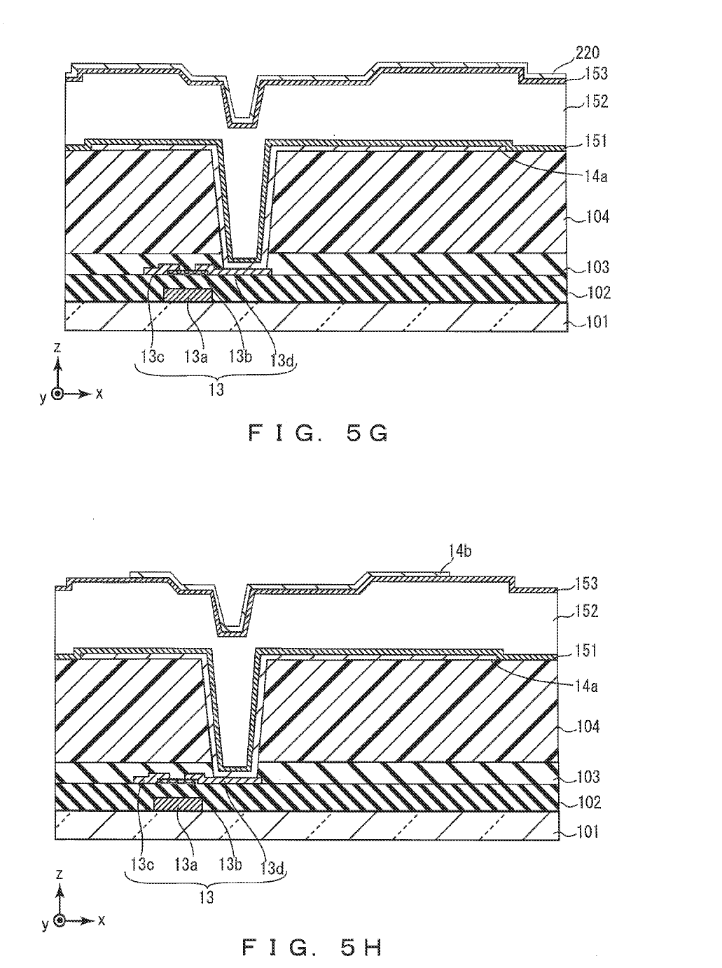

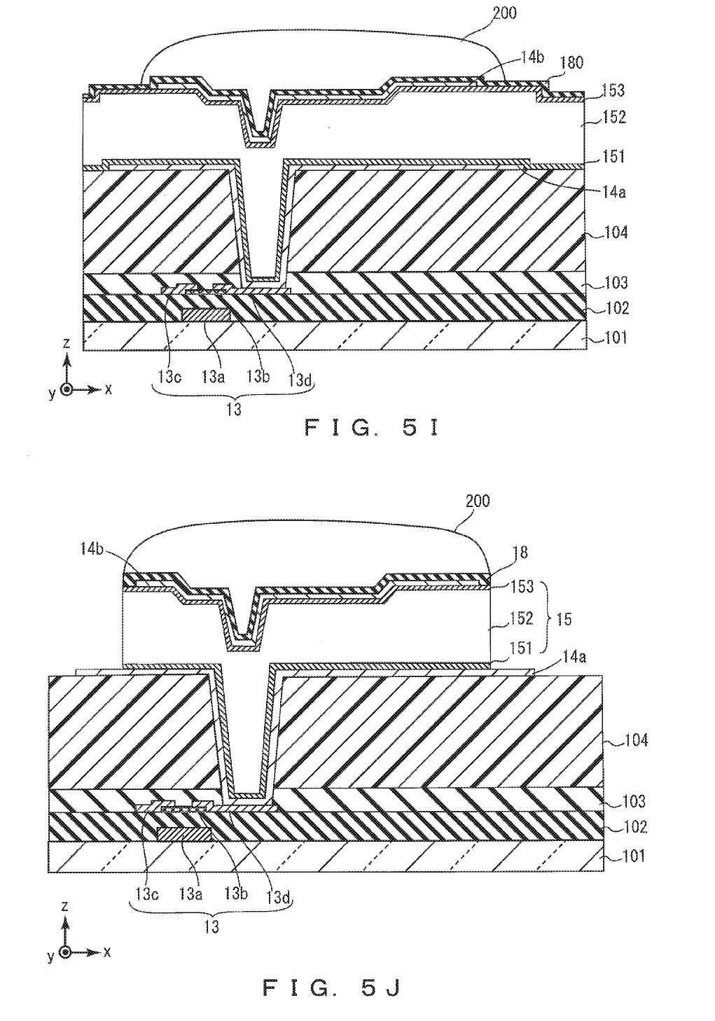

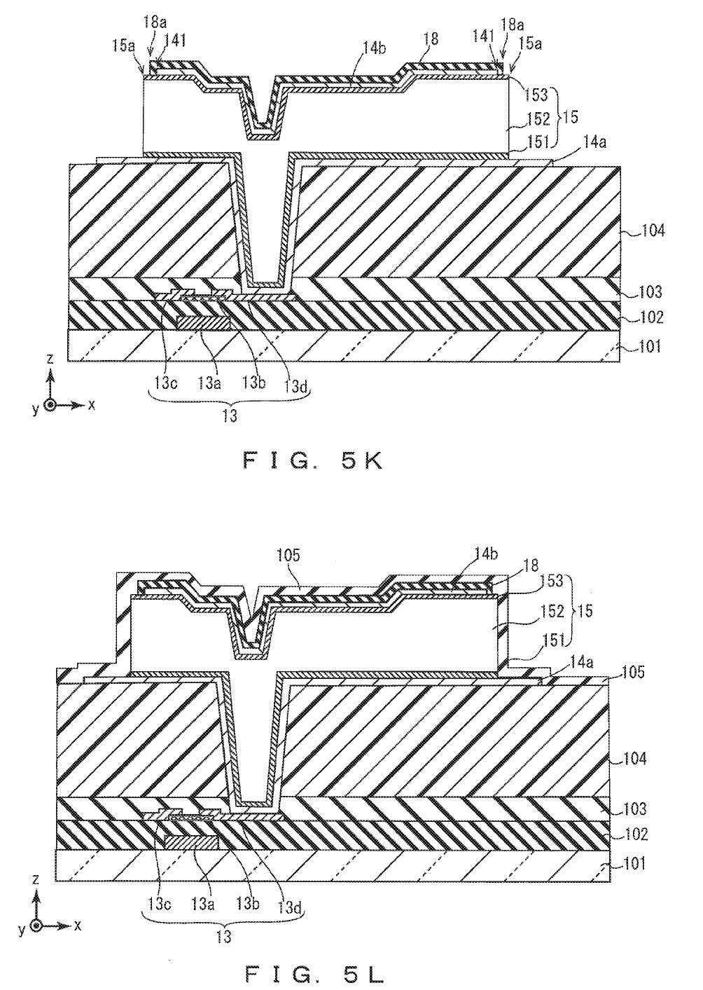

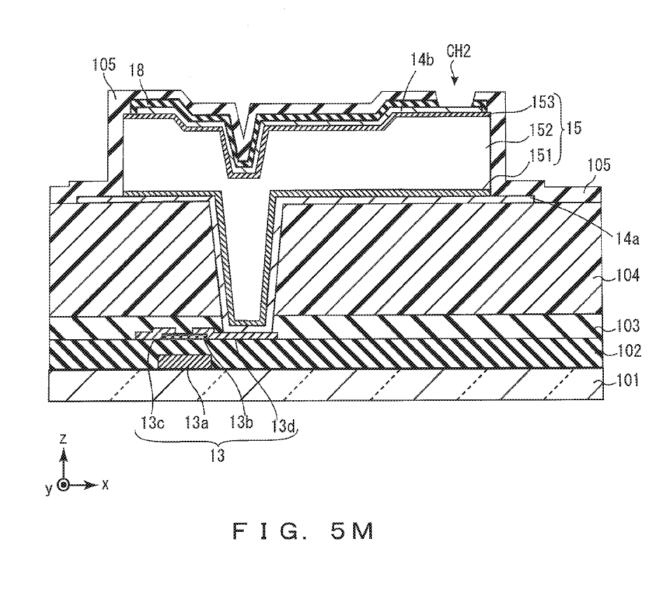

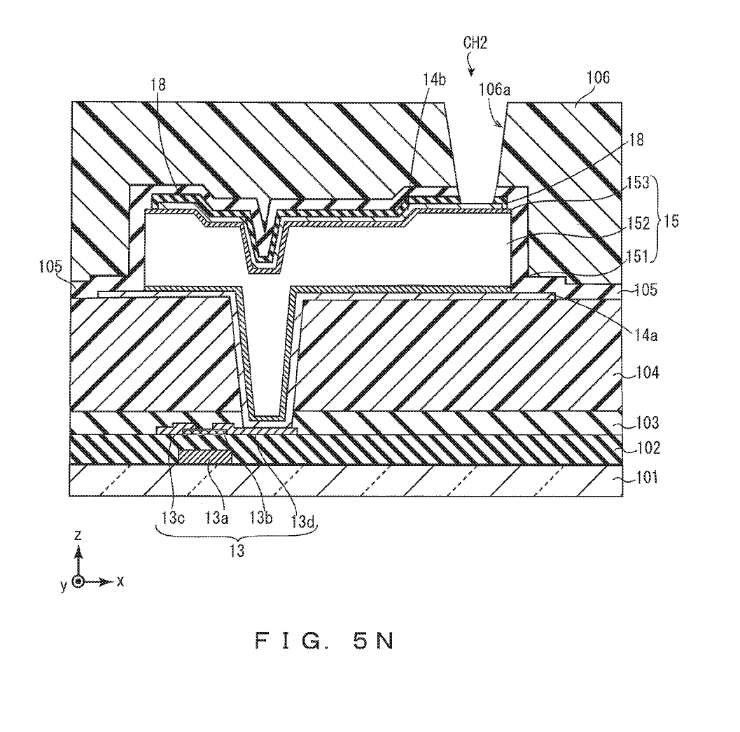

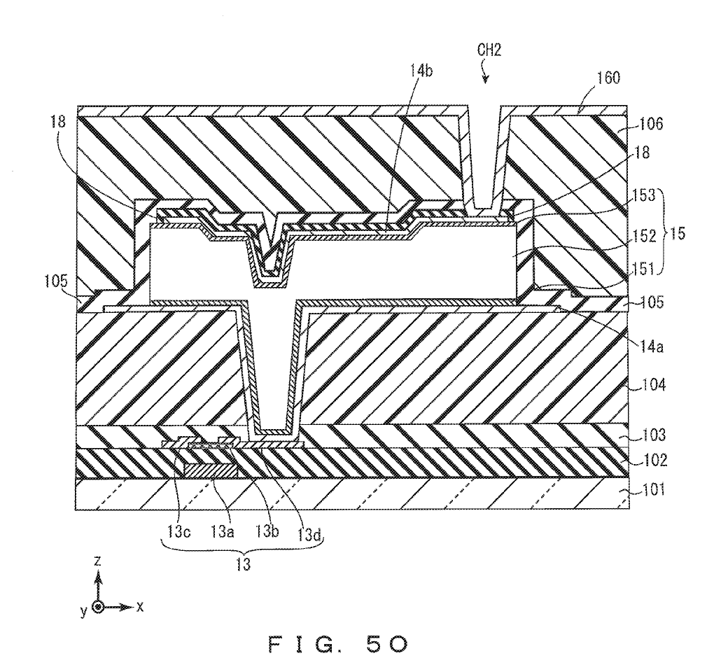

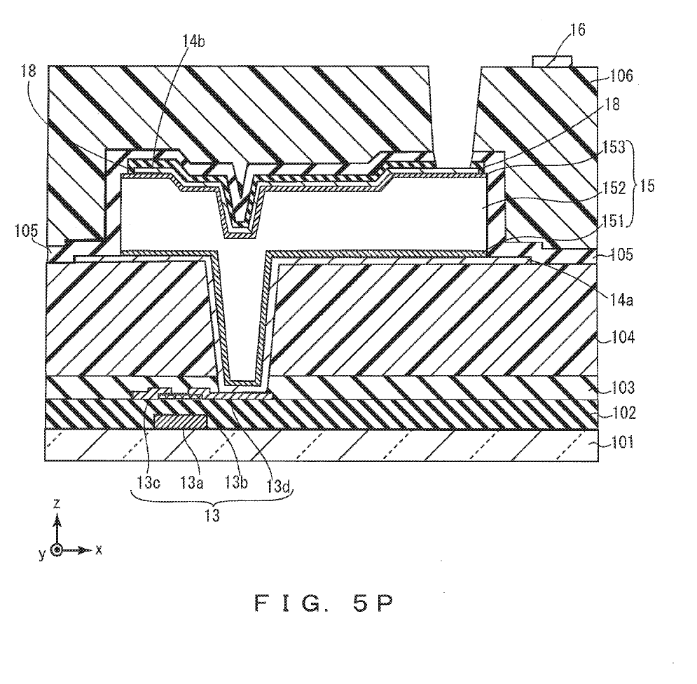

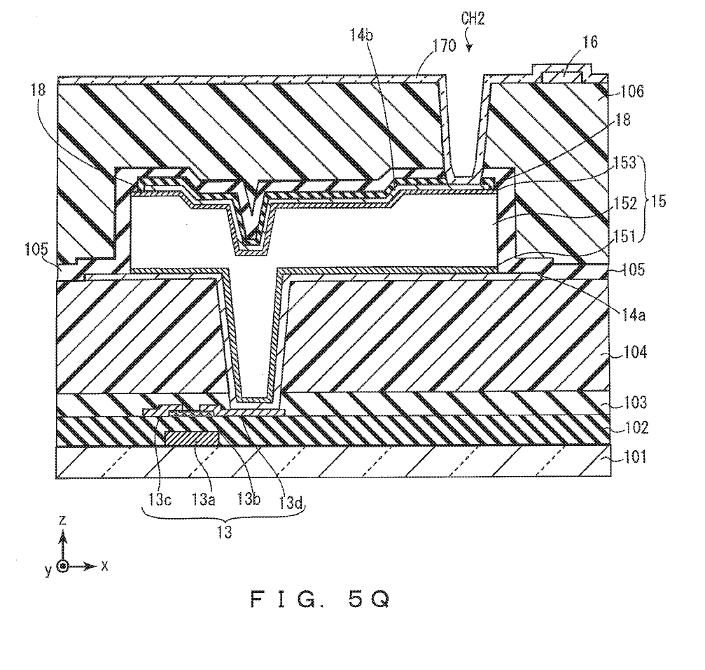

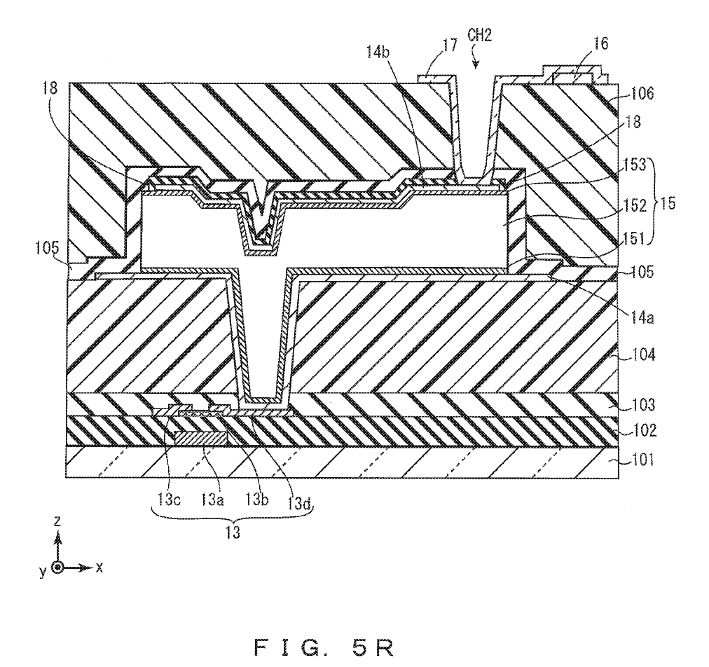

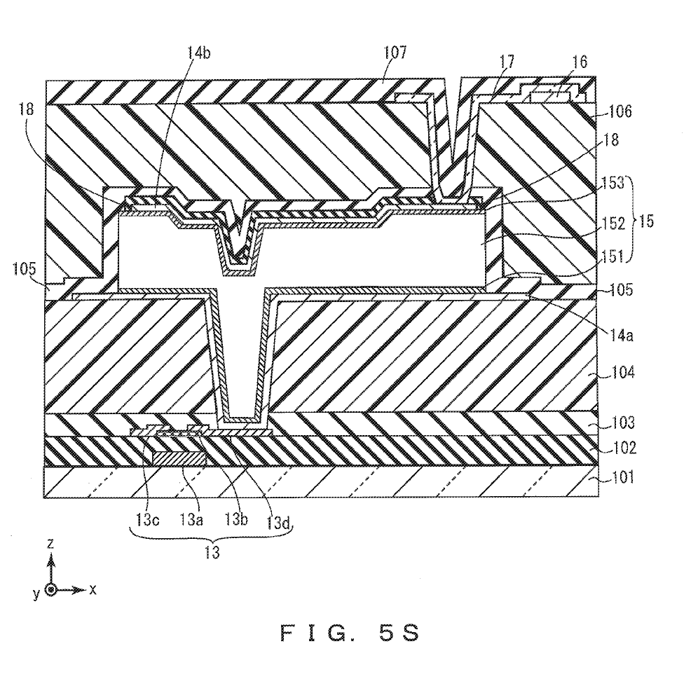

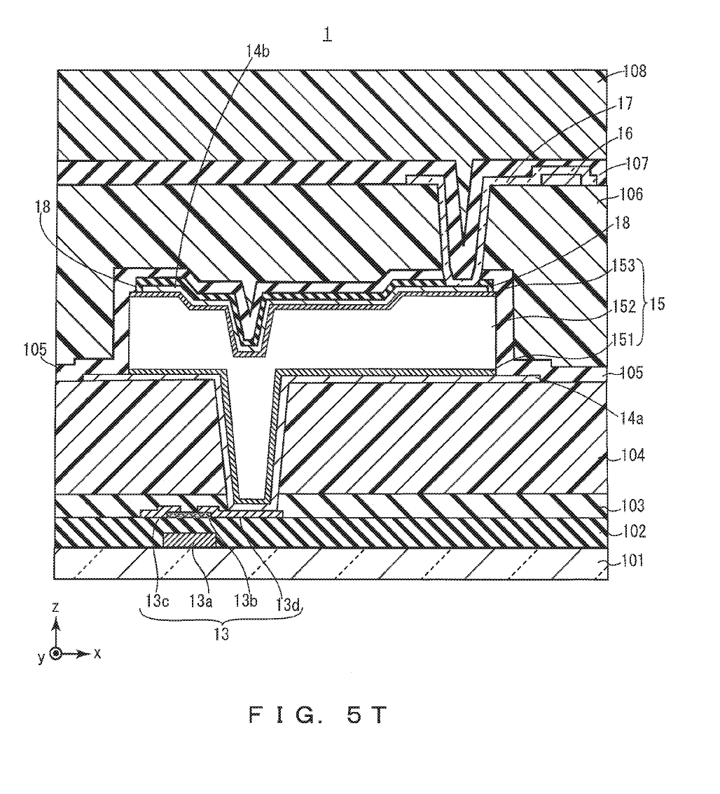

Next, the following description describes a method for producing the imaging panel 1. FIGS. 5A to 5T are cross-sectional views of the pixel taken along line A-A in respective steps of the method for producing the imaging panel 1 (see FIG. 3).

As illustrated hi FIG. 5A, the gate insulating film 102 and the TFT 13 are formed on the substrate 101 by a known method, and the first insulating film 103 made of silicon nitride (SiN) is formed by, for example, plasma CVD, so as to cover the TFT 13.

Subsequently, a heat treatment at about 350° C. is applied to an entire surface of the substrate 101, and photolithography and wet etching are carried out so that the first insulating film 103 is patterned, whereby the contact hole CH1 is formed on the drain electrode 13d (see FIG. 5B).

Next, the second insulating film 104 made of acrylic resin or siloxane-based resin is formed on the first insulating film 103 by, for example, slit coating (see FIG. 5C).

An opening 104a of the second insulating film 104 is formed by photolithography on the contact hole CH1 (see FIG. 5D).

Subsequently, a metal film 210 made of molybdenum nitride (MoN) is formed on the second insulating film 104 by, for example, sputtering (see FIG. 5E).

Then, photolithography and wet etching are carried out, whereby the metal film 210 is patterned. Through these steps, the lower electrode 14a, which is connected with the drain electrode 13d through the contact hole CH1, is formed on the second insulating film 104 (see FIG. 5F).

Next, the n-type amorphous semiconductor layer 151, the intrinsic amorphous semiconductor layer 152, and the p-type amorphous semiconductor layer 153 are formed in this order on the second insulating film 104 by, for example, plasma CVD, so as to cover the lower electrode 14a. Then, a transparent conductive film 220 made of, for example, ITO is formed on the p-type amorphous semiconductor layer 153 (see FIG. 5G).

Thereafter, photolithography and dry etching are carried out so that the transparent conductive film 220 is patterned, whereby the upper electrode 14b is formed on the p-type amorphous semiconductor layer 153 (see FIG. 5H).

Subsequently, an insulating film 180 made of silicon nitride (SiN) is formed on the p-type amorphous semiconductor layer 153 by, for example, plasma CVD, so as to cover the upper electrode 14b. Then, a resist 200 is applied on the insulating film 180 (see FIG. 5I).

Then, photolithography and dry etching are carried out, whereby the insulating film 180, the n-type amorphous semiconductor layer 151, the intrinsic amorphous semiconductor layer 152, and the p-type amorphous semiconductor layer 153 are patterned. Through these steps, the photoelectric conversion layer 15 and the upper electrode protection film 18, having smaller widths in the X-axis direction than the width of the lower electrode 14a, are formed (see FIG. 5J).

Next, the resist 200 is removed, and thereafter, in order to suppress leakage current in the photoelectric conversion layer 15, a reduction treatment using hydrogen fluoride is applied to the surfaces of the upper electrode protection film 18 and the photoelectric conversion layer 15. The upper electrode protection film 18 is partially etched in the X-axis direction by the reduction treatment. As a result, each end 18a of the upper electrode protection film 18 is arranged between the X-axis direction end 141 of the upper electrode 14b and the end 15a of the photoelectric conversion layer 15 (see FIG. 5K).

In this way, the upper electrode protection film 18 is partially etched in the X-axis direction by the reduction treatment using hydrogen fluoride, but the upper electrode 14b is not exposed to hydrogen fluoride since it is covered with the upper electrode protection film 18. The reduction treatment using hydrogen fluoride does not lead to a phenomenon that metal ions generated as a result of dissolution of the upper electrode 14b would adhere to side surfaces of the photoelectric conversion layer 15.

Next, the third insulating film 105 made of silicon nitride (SiN) is formed on the upper electrode protection film 18 by, for example, plasma CVD (see FIG. 5L).

Then, photolithography and wet etching are carried out so that the contact hole CH2 passing through the third insulating film 105 and the upper electrode protection film 18 is formed (see FIG. 5M).

Subsequently, the fourth insulating film 106 made of acrylic resin or siloxane-based resin is formed on the third insulating film 105 by, for example, slit coating. Then, an opening 106a in the fourth insulating film 106 is formed by photolithography on the contact hole CH2 (see FIG. 5N).

Next, a metal film 160 is formed by laminating molybdenum nitride (MoN), aluminum (Al), and titanium (Ti) in this order on the fourth insulating film 106 by, for example, sputtering (see FIG. 5O).

Then, photolithography and wet etching are carried out so that the metal film 160 is patterned, whereby the bias line 16 is formed (see FIG. 5P).

Subsequently, a transparent conductive film 170 made of ITO is formed by, for example, sputtering on the fourth insulating film 106 so as to cover the bias line 16 (see FIG. 5Q).

Then, photolithography and dry etching are carried out so that the transparent conductive film 170 is patterned, whereby the transparent conductive film 17 is formed that is connected with the bias line 16 and is connected with the upper electrode 14b through the contact hole CH2 (see FIG. 5R).

Next, the fifth insulating film 107 made of silicon nitride (SiN) is formed by, for example, plasma CVD on the fourth insulating film 106 so as to cover the transparent conductive film 17 (see FIG. 5S).

Subsequently, the sixth insulating film 108 made of acrylic resin or siloxane-based resin is formed on the fifth insulating film 107 by, for example, slit coating (see FIG. 5T).

What is described above is the method for producing the imaging panel 1 in the present embodiment. As described above, the upper electrode protection film 18 is formed on the upper electrode 14b of the photodiode 12. In this configuration, the upper electrode 14b is thus covered with the upper electrode protection film 18, which results in the following: even if a reduction treatment using hydrogen fluoride is carried out after the resist 200 used for forming the photodiode 12 (see FIG. 5J) is removed, the upper electrode 14b is not exposed to hydrogen fluoride, and metal ions of the upper electrode 14b do not adhere to the side surfaces of the photoelectric conversion layer 15. Further, since a reduction treatment using hydrogen fluoride is carried out after the resist 200 is removed, it is less likely that organic substances would adhere to the side surfaces of the photoelectric conversion layer 15, as compared with the case where the resist 200 is removed after a reduction treatment using hydrogen fluoride. This makes it possible to prevent the side surfaces of the photoelectric conversion layer 15 from being contaminated with metals or organic substances, thereby to suppress leakage current in the photodiode 12.

### (Operation of X-Ray Imaging Device 100)

Here, operations of the X-ray imaging device 100 illustrated in FIG. 1 are described. First, X-rays are emitted from the X-ray source 3. Here, the control unit 2 applies a predetermined voltage (bias voltage) to the bias line 16 (see FIG. 3 and the like). X-rays emitted from the X-ray source 3 are transmitted through an object S, and are incident on the scintillator 1A. The X-rays incident on the scintillator 1A are converted into fluorescence (scintillation light), and the scintillation light is incident on the imaging panel 1. When the scintillation light is incident on the photodiode 12 provided in each pixel in the imaging panel 1, the scintillation light is changed to charges by the photodiode 12 in accordance with the amount of the light. A signal according to the charges obtained by conversion by the photodiode 12 is read out through the source line 10 to the signal reading unit 2B (see FIG. 2 and the like) when the TFT 13 (see FIG. 3 and the like) is in the ON state according to a gate voltage (positive voltage) that is output from the gate control unit 2A through the gate line 11. Then, an X-ray image in accordance with the signal thus read out is generated in the control unit 2.

### Embodiment 2

Embodiment 1 is described above with reference to an exemplary case where in the step illustrated in FIG. 5K, after the resist 200 (see FIG. 5J) is removed, a reduction treatment using hydrogen fluoride is carried out, and thereafter, in the step illustrated in FIG. 5L, the third insulating film 105 is formed. The process, however, may be as follows.

In the above-described step illustrated in FIG. 5K, after a reduction treatment using hydrogen fluoride is carried out, before the third insulating film 105 is formed, the surfaces of the upper electrode protection film 18 and the photoelectric conversion layer 15 are subjected to a hydrogen-gas-containing plasma treatment.

By performing a hydrogen-gas-containing plasma treatment subsequently to a reduction treatment using hydrogen fluoride in this way, the effect of suppressing leakage current in the photodiode 12 can be further improved as compared with Embodiment 1.

Besides, in a case where the upper electrode protection film 18 is not provided, when a hydrogen-gas-containing plasma treatment is applied to the surface of the photodiode 12, metals contained in the upper electrode 14b are reduced by the plasma treatment, whereby the transmittance of the upper electrode 14b decreases. In the present embodiment, the upper electrode 14b is covered with the upper electrode protection film 18. Even if a hydrogen-gas-containing plasma treatment is carried out before the third insulating film 105 is formed, the upper electrode 14b therefore is not affected by the plasma treatment, and the transmittance is not caused to decrease, which results in that it is unlikely that the light receiving sensitivity of the photodiode 12 would decrease.

### Embodiment 3

Embodiment 1 and Embodiment 2 are described above with reference to an exemplary case where a reduction treatment using hydrogen fluoride is carried out in the step illustrated in FIG. 5K. In the present embodiment, a hydrogen-gas-containing plasma treatment is carried out in place of the reduction treatment using hydrogen fluoride.

In other words, after the step illustrated in FIG. 5J, the resist 200 is removed, and a hydrogen-gas-containing plasma treatment is carried out. Thereafter, by the step illustrated in FIG. 5L, the third insulating film 105 is formed on the upper electrode protection film 18. By carrying out the hydrogen-gas-containing plasma treatment in this way, leakage current on the surface of the photoelectric conversion layer 15 can be suppressed. Besides, in the present embodiment as well, the upper electrode 14b is covered with the upper electrode protection film 18. Even if a hydrogen-gas-containing plasma treatment is carried out before the third insulating film 105 is formed, the upper electrode 14b therefore is not affected by the plasma treatment, and the transmittance is not caused to decrease, which results in that it is unlikely that the light receiving sensitivity of the photodiode 12 would decrease.

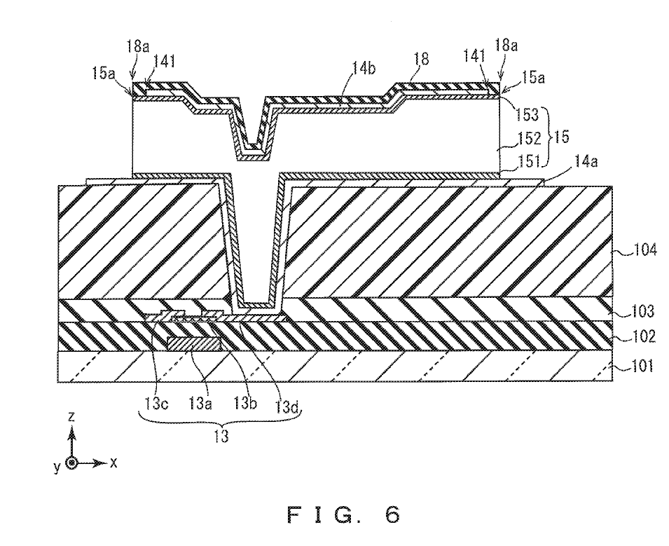

Incidentally, in a case where a reduction treatment using hydrogen fluoride is carried out in the step illustrated in FIG. 5K, as described above, a part of the upper electrode protection film 18 is etched in the X-axis direction, and the position of the end 18a of the upper electrode protection film 18 is arranged on an inner side with respect to the end 15a of the photoelectric conversion layer 15. On the other hand, in a case where the resist 200 is removed after the step illustrated in FIG. 5J and a hydrogen-gas-containing plasma treatment is carried out, the ends of the upper electrode protection film 18 are not etched. As a result, in this case, as illustrated in FIG. 6, each end 18a of the upper electrode protection film 18 is arranged at approximately the same position as the position of the end 15a of the photoelectric conversion layer 15.

The embodiments of the present invention, described above, are merely examples for implementing the present invention. The present invention, therefore, is not limited to the above-described embodiments, but can be appropriately modified without deviating from the scope of the invention and be implemented. The following description describes modifications of the present invention.

(1) Embodiments 1 to 3 are described above with reference to an exemplary case where silicon nitride (SiN) is used as a material for the upper electrode protection film 18, but silicon oxide (SiO₂) may be replaced with silicon nitride (SiN), or alternatively, silicon oxide nitride (SiON) may be used.

Silicon nitride (SiN), silicon oxide (SiO₂), and silicon oxide nitride (SiON) provide different adhesivenesses with the upper electrode 14b, respectively, when they are used for forming the upper electrode protection film 18. More specifically, the respective adhesivenesses with the upper electrode 14b of silicon nitride (SiN), silicon oxide (SiO₂), and silicon oxide nitride (SiON) descend in this order. In a case where the adhesiveness with the upper electrode 14b is taken into consideration, therefore, it is preferable to use silicon nitride (SiN) as a material for the upper electrode protection film 18.

Further, silicon nitride (SiN), silicon oxide (SiO₂), and silicon oxide nitride (SiON) are etched to different levels by a reduction treatment using hydrogen fluoride, respectively. In other words, the relationship of the etched amounts of silicon nitride (SiN), silicon oxide (SiO₂), and silicon oxide nitride (SiON) in a reduction treatment using hydrogen fluoride is as follows: silicon nitride (SiN)<silicon oxide (SiO₂)<silicon oxide nitride (SiON). The upper electrode protection film 18 after a reduction treatment using hydrogen fluoride is carried out preferably has a thickness of 70 μm or more. The thickness of the film when the film is formed is therefore set according to the material used for forming the upper electrode protection film 18. For example, the films of silicon nitride (SiN), silicon oxide (SiO₂), and silicon oxide nitride (SiON) are formed so as to have thicknesses of 100 nm, 150 nm, and 200 nm, respectively when the films are just formed.

1. An imaging panel that generates an image based on scintillation light that is obtained from X-rays transmitted through an object, the imaging panel comprising:
a substrate;
a thin film transistor that is formed on the substrate;
an insulating film that covers the thin film transistor;
a photoelectric conversion layer that is provided on the insulating film, and converts the scintillation light into charges;
an upper electrode that is provided on the photoelectric conversion layer;
a lower electrode that is provided under the photoelectric conversion layer, and is connected with the thin film transistor; and
an upper electrode protection film that covers the upper electrode, above the photoelectric conversion layer,
wherein ends of the upper electrode are arranged in such a manner that each end thereof is arranged on an inner side of the photoelectric conversion layer with respect to a corresponding end of the photoelectric conversion layer, and
wherein ends of the upper electrode protection film are arranged in such a manner that each end thereof is arranged between a corresponding end of the upper electrode and a corresponding end of the photoelectric conversion layer.

2. The imaging panel according to claim 1,
wherein the upper electrode protection film is made of silicon nitride.

3. The imaging panel according to claim 1,
wherein the upper electrode protection film is made of silicon oxide.

4. The imaging panel according to claim 1,
wherein the upper electrode protection film is made of silicon oxide nitride.

5. A method for producing an imaging panel that generates an image based on scintillation light that is obtained from X-rays transmitted through an object, the producing method comprising:
forming a thin film transistor on a substrate;
forming a first insulating film and a second insulating film on the thin film transistor;
forming a first contact hole on a drain electrode of the thin film transistor so that the first contact hole passes through the first insulating film and the second insulating film;
forming, on the second insulating film, a first transparent electrode film as a lower electrode that is connected with the drain electrode through the first contact hole;
forming a first semiconductor layer of a first conductive type, an intrinsic amorphous semiconductor layer, and a second semiconductor layer of a second conductive type that is opposite to the first conductive type, in the stated order, as a photoelectric conversion layer on the first transparent electrode film;
forming an upper electrode on the second semiconductor layer;
forming an insulating film as an upper electrode protection film, on the upper electrode;
applying a resist on the insulating film, and etching the insulating film, the first semiconductor layer, the intrinsic amorphous semiconductor layer, and the second semiconductor layer, so as to form the photoelectric conversion layer and the upper electrode protection film;
removing the resist, and thereafter, carrying out a reduction treatment with respect to a surface of the photoelectric conversion layer;
forming a third insulating film that covers the upper electrode protection film, after the reduction treatment;
forming a second contact hole on the upper electrode so that the second contact hole passes through the third insulating film and the upper electrode protection film;
forming a fourth insulating film on the third insulating film except for a portion of the second contact hole;
forming a signal line for supplying a bias voltage, on the fourth insulating film;
forming, on the fourth insulating film, a transparent conductive film that connects the signal line and the upper electrode with each other through the second contact hole; and
forming a fifth insulating film that covers the transparent conductive film.

6. The producing method according to claim 5,
wherein, as the reduction treatment, a reduction treatment using hydrogen fluoride is carried out.

7. The producing method according to claim 6,
wherein, after the reduction treatment using hydrogen fluoride is carried out, before the third insulating film is formed, a hydrogen-gas-containing plasma treatment is carried out.

8. The producing method according to claim 5,
wherein, as the reduction treatment, a reduction treatment using hydrogen gas is carried out.
